(19) United States
(12) Patent Application Publication
(10) Pub. No.: US 2024/0150278 A1
(43) Pub. Date: May 9, 2024

(54) PROCESS FOR PURIFYING AMINOCAPRONITRILE

(71) Applicant: Ascend Performance Materials Operations LLC, Houston, TX (US)

(72) Inventors: C. Alex Diaz, Houston, TX (US); Mikhail I. Khramov, Houston, TX (US); Scott G. Moffatt, Houston, TX (US)

(73) Assignee: Ascend Performance Materials Operations LLC, Houston, TX (US)

(21) Appl. No.: 18/501,318

(22) Filed: Nov. 3, 2023

Related U.S. Application Data
(60) Provisional application No. 63/422,814, filed on Nov. 4, 2022.

(51) Int. Cl. C07C 253/34; C07C 253/22

(52) U.S. Cl. CPC C07C 253/34; C07C 253/22

(57) ABSTRACT

A process for converting caprolactam to aminocapronitrile (ACN), the process comprising contacting a caprolactam feed stream with ammonia to produce a first crude product stream; separating the first crude product stream to produce an intermediate product stream comprising ACN and caprolactam recovery stream; and purifying the intermediate product stream to produce a purified product stream comprising greater than 95 wt % aminocapronitrile.

### CROSS-REFERENCE TO RELATED APPLICATION

This application claims priority from U.S. Provisional Application No. 63/422,814 entitled “Process For Purifying Aminocapronitrile” filed Nov. 4, 2022, the disclosure of which in incorporated herein by reference in its entirety.

### FIELD

The present disclosure provides a process for purifying aminocapronitrile (ACN) produced from caprolactam. Specifically, the present disclosure provides a process, e.g., gas-phase and/or aqueous phase, for converting caprolactam to crude aminocapronitrile (ACN). The crude ACN can be purified in an initial process step such as a quench column at a pressure greater than 0.101 MPa to provide high purity ACN. The ACN thus purified may then be further reacted, for example, to produce hexamethylenediamine (HMD).

### BACKGROUND

Methods for the manufacture and purification of 6-aminocapronitrile (ACN) are known. ACN is a valuable chemical species, which may be used, for example, to produce hexamethylenediamine (HMD). HMD itself may be further reacted to produce polyamides, such as PA6,6; PA 6,10; and PA6,12. It may also be used to produce hexamethylene diisocyanate, which may be used in the production of polyurethanes, for example.

The purification of ACN produced from caprolactam may be problematic due to the need to separate ACN from impurities produced during the reaction with boiling points close to that of ACN. Conventional processes often rely on expensive and energy-intensive processes, such as batch vacuum distillation, to produce high purity ACN.

CN111233704A discloses a method for preparing a 6-aminocapronitrile product, which comprises the following steps: a) uniformly mixing caprolactam, an acid solution and the like according to a certain proportion, adding the mixture into a reaction kettle, heating and stirring the mixture for reaction; b) after the caprolactam in the step A is reacted, removing low-boiling-point substances under reduced pressure, and recrystallizing to obtain 6-aminocaproic acid salt; c) uniformly mixing the 6-aminocaproate, the alcohol, the ammoniating agent, the dehydrating agent and the like obtained in the step B according to a certain proportion, adding the mixture into a reaction kettle, heating and stirring the mixture for reaction; d) rectifying the reaction liquid obtained in the step C, separating and purifying to obtain the 6-aminocapronitrile.

CN112094202A discloses a method for circularly synthesizing a key intermediate of hexamethylene diamine, which comprises the steps of carrying out ammoniation reaction of caprolactam in two sections, simultaneously returning kettle residues generated in the process of preparing 6-aminocapronitrile by catalyzing and ammoniating the caprolactam to the second section of ammoniation reaction, hydrolyzing the kettle residues into caprolactam and 6-aminocapronitrile by using water generated in the first section of ammoniation reaction and unreacted ammonia gas at high temperature, and carrying out ammoniation reaction on the obtained caprolactam in a reactor to generate the 6-aminocapronitrile.

Even in view of the known processes, the need exists for processes for producing high purity ACN comprising low quantities of impurities, e.g., caprolactam, aminocaproic acid, cyclohexanone, cyclohexenone, n-hexanenitrile, 5-hexenenitrile, caprolactam dimers, caprolactam oligomers, N-phenylacetamide, without the need for a low pressure, e.g., vacuum, separation steps.

### SUMMARY

The present disclosure provides a process for converting caprolactam to aminocapronitrile (ACN). The process comprises contacting a caprolactam feed stream with ammonia to produce a first crude product stream, separating the first crude product stream, e.g., via a (continuous) distillation at a pressure at a pressure greater than 0.101 MPa and/or less than 100 kPa, to produce an intermediate product stream comprising ACN and an ammonia recovery stream comprising ammonia, and purifying the intermediate product stream to produce a purified product stream comprising greater than 95 wt % aminocapronitrile, e.g., greater than 98 wt %. The caprolactam feed stream comprises less than 99 wt. % caprolactam and greater than 1 wt. % impurities. The contacting step is conducted in a reactor, preferably a fixed bed reactor. The separating is conducted at a pressure ranging from 0.101 MPa to 0.120 MPa, and a temperature at the top of the column ranging from 60° C. to 90° C., e.g., 75° C. to 80° C. The separating comprises quenching the first crude product to produce a vapor ammonia recovery stream, a liquid ammonia recovery stream, and a first intermediate product stream. The vapor ammonia recovery stream comprises greater than 95 wt % ammonia and less than 5 wt % water; the liquid ammonia recovery stream comprises greater than 20 wt % ammonia, and the intermediate product stream comprises heavies and greater than 40 wt % aminocapronitrile. The vapor ammonia recovery stream is compressed, preferably via a centrifugal compressor, to form a compressed vapor ammonia recovery stream. The liquid ammonia recovery stream is further separated to produce a supplemental ammonia recovery stream comprising ammonia and a bottoms stream comprising water. At least a portion of the vapor ammonia stream, the liquid ammonia recovery stream, and/or supplemental ammonia recovery stream may be recycled to the ammonia feed stream or to the contacting step. In some cases, the residence time in any one column during distillation is less than 180 minutes. In some cases, the recovery of ACN in the intermediate product stream is greater than 70%, and/or the recovery of caprolactam in the caprolactam recovery stream is greater than 90%.

The purifying step comprises separating the intermediate product stream to produce a first purified product stream and a crude column bottoms. The first purified product stream comprises greater than 95 wt. % aminocapronitrile and less than 5 wt. % of impurities comprising cyclohexanone, cyclohexenone, n-hexanenitrile, 5-hexenenitrile, aminocaproic acid, caprolactam, caprolactam dimers caprolactam oligomers, or n-phenylacetamide, or combinations thereof. The first purified product stream may be further separated to form a second purified product stream comprising greater than 95 wt % aminocapronitrile, preferably greater than 99 wt % aminocapronitrile. The second purified product stream may be taken as a side-draw. The crude column bottoms is separated to produce a distillate caprolactam recycle stream comprising greater than 95 wt % caprolactam and heavy byproducts stream containing organic impurities and less than 10 wt % caprolactam. The caprolactam recycle stream may be recycled to the caprolactam feed stream or to the reaction step.

### BRIEF DESCRIPTION OF THE DRAWING

FIG. 1 shows a schematic of a method for the production and purification of aminocapronitrile (ACN).

### DETAILED DESCRIPTION

### Introduction

As noted above, conventional processes for the purification of aminocapronitrile (ACN), e.g., 6-ACN, generally utilize separation schemes that comprise an initial process step, e.g., a quenching step, that require low-pressure (as low as 1 kPa (0.001 MPa) or even vacuum) operation. Low pressure operation requires significant equipment and energy, both to achieve the low pressure and to then compress and recycle unreacted ammonia that results from the separation. This vacuum operation typically requires specific, high-performance equipment to withstand these operating conditions, which incurs significant expense.

Also, the conventional lower pressure distillation processes create downstream and/or recycling challenges, e.g., in the subsequent reaction of the caprolactam with recycled ammonia to form ACN. Because of the vacuum operation of the initial step, e.g., the quenching, the separated ammonia is at a low pressure. And to conduct the reaction efficiently and avoid wasting resources, the ammonia is often conveyed/recycled back to the caprolactam-to-ACN reaction step. However, in order to be effectively utilized in the reaction, the ammonia must be compressed, e.g., by passing the ammonia stream through a compressor. The low pressure operation of the quench step contributes to the need for compression and ultimately creates a significant burden on the recycle ammonia compressor.

The inventors have found that operating an initial separation (quenching) step at higher pressures, e.g., pressures closer to atmospheric pressure, operational efficiencies are significantly improved. As one example, higher pressure quenching advantageously reduces the load on pumps and compressors for feeding or recycling ammonia, thereby lowering operating costs. In some cases, it has been found that by operating the quench column at atmospheric pressure, the resultant overhead vapor (ammonia and water inter alia) can be effectively condensed, e.g., in an overhead partial condenser that operates at lower size and energy consumption levels, to separate the ammonia from the water. In particular, the disclosed process advantageously reduces the pressure ratio of the recycle compressor, which may further contribute to the aforementioned benefits.

Importantly, in some embodiments, the processes disclosed herein may operate in a continuous manner. It has been found that by doing so, the processes surprisingly limit losses in yield. Additionally, it has been found that low residence times and lower temperatures may minimize loss of aminocapronitrile to undesired side reactions. Unexpectedly, continuous operation may also increase recovery of caprolactam, thereby further lessening waste, versus conventional batch operation. Without being bound by theory, it is postulated that less degradation of both ACN and caprolactam may occur during continuous operation, such as continuous distillation. It is thought that the shorter residence times in comparison to conventional batch distillation results in fewer undesirable side reactions which lead to decreases in recovery of both ACN and caprolactam.

In addition, it has been discovered that the disclosed process provides the additional advantage of being able to utilize caprolactam feed streams that are not be of high purity, as is conventional. Traditionally, the formation of high purity streams often requires significant processing, which may be costly and complicated. When the disclosed process is employed, lower purity caprolactam feed streams may beneficially be used and similar, if not improved, reaction parameters are demonstrated.

### Separation of the First Crude Product

The present disclosure relates to a process for converting caprolactam to ACN. The process comprises the step of contacting a caprolactam feed stream with ammonia to produce a crude product stream comprising ACN and ammonia. The process further comprises the step of separating, e.g., quenching, the first crude product stream at a higher pressure, higher than conventional separation operations, to produce an intermediate stream comprising ACN and an ammonia recovery stream comprising ammonia. The intermediate stream is then purified to produce a purified product stream comprising high purity ACN. The ammonia recovery stream is recycled back into the reaction.

### Quench Column

In some embodiments, the first crude product stream is separated, e.g., quenched, in a (quench) column, to produce a vapor ammonia recovery stream and/or a liquid ammonia recovery stream. The separation step may also produce the intermediate (ACN) stream.

In some embodiments, the (quench) column is operated at pressure ranging from 0.101 MPa to 0.150 MPa, e.g., from 0.105 MPa to 0.145 MPa, from 0.110 MPa to 0.140 MPa, from 0.115 MPa to 0.135 MPa, or from 0.120 MPa to 0.130 MPa. In terms of upper limits, the column is operated at a pressure less than 0.150 MPa, e.g., less than 0.145 MPa, less than 0.140 MPa, less than 0.135 MPa, or less than 0.130 MPa. In terms of lower limits, the column is operated at a pressure greater than 0.101 MPa, e.g., greater than 0.105 MPa, greater than 0.110 MPa, greater than 0.115 MPa, or greater than 0.120 MPa. As noted above, operation at this pressure provides for the aforementioned benefits. Further, these pressures contribute to an increase in the ammonia concentration in the vapor ammonia recovery stream (the liquid distillate), which has advantageous effects on the downstream separation steps, e.g., improving the energy efficiency of the process.

In an embodiment, the (quench) column is operated at a pressure ranging from 0.101 MPa to 0.120 MPa.

In some cases, the compression ratio as the ammonia vapor is passed through the compressor ranges from 1 to 4, e.g., from 1.2 to 3.8, from 1.4 to 3.6, from 1.6 to 3.4, from 1.8 to 3.2, from 2.0 to 3.0, from 2.2 to 2.8, or from 2.4 to 2.6. In terms of upper limits, the compression ratio is less than 4; e.g., less than 3.9, less than 3.8, less than 3.7, less than 3.6, less than 3.5, less than 3.4, less than 3.3, less than 3.2, less than 3.1, less than 3.0, less than 2.9, less than 2.8, less than 2.7, less than 2.6, or less than 2.5. In terms of lower limits, the compression ratio is greater than 1, e.g., greater than 1.1, greater than 1.2, greater than 1.3, greater than 1.4, greater than 1.5, greater than 1.6, greater than 1.7, greater than 1.8, greater than 1.9, greater than 2.0, greater than 2.1, greater than 2.2, greater than 2.3, or greater than 2.4.

The suction pressure of the ammonia vapor (prior to passing through the compressor) may range from 0.100 MPa to 1 MPa, e.g., from 0.200 MPa to 0.900 MPa, from 0.300 MPa to 0.800 MPa, from 0.400 MPa to 0.700 MPa, or from 0.500 MPa to 0.600 MPa. In some embodiments, the starting pressure of the ammonia vapor prior to passing through the compressor may range from 0.100 MPa to 0.200 MPa, 0.101 MPa to 0.199 MPa, from 0.105 MPa to 0.195 MPa, from 0.110 MPa to 0.190 MPa, from 0.115 MPa to 0.185 MPa, from 0.120 MPa to 0.180 MPa, from 0.125 MPa to 0.175 MPa, from 0.130 MPa to 0.170 MPa, from 0.135 MPa to 0.165 MPa, from 0.140 MPa to 0.160 MPa, or from 0.145 to 0.155 MPa. In terms of upper limits, the starting pressure of the ammonia vapor prior to passing through the compressor may be less than 1 MPa, e.g., less than 0.900 MPa, less than 0.800 MPa, less than 0.700 MPa, or less than 0.600 MPa. In terms of lower limits, the starting pressure of the ammonia vapor prior to passing through the compressor may be greater than 0.101 MPa, e.g., greater than 0.105 MPa, greater than 0.110 MPa, greater than 0.115 MPa, greater than 0.120 MPa, greater than 0.130 MPa, greater than 0.135 MPa, greater than 0.140 MPa, greater than 0.145 MPa, greater than 0.150 MPa, greater than 0.155 MPa, greater than 0.160 MPa, greater than 0.165 MPa, greater than 0.170 MPa, greater than 0.175 MPa, greater than 0.180 MPa, greater than 0.185 MPa, greater than 0.190 MPa, greater than 0.199 MPa, greater than 0.200 MPa, greater than 0.300 MPa, greater than 0.400 MPa, or greater than 0.500 MPa.

The discharge pressure of the ammonia vapor (after passing through the compressor) may range from 0.201 MPa to 0.405 MPa, e.g., from 0.202 MPa to 0.404 MPa, from 0.205 MPa to 0.400 MPa, from 0.210 MPa to 0.390 MPa, from 0.220 MPa to 0.380 MPa, from 0.240 MPa to 0.370 MPa, from 0.250 MPa to 0.360 MPa, from 0.260 MPa to 0.350 MPa, from 0.270 MPa to 0.340 MPa, from 0.280 MPa to 0.320 MPa, or from 0.290 to 0.310 MPa. In terms of upper limits, the final pressure of the ammonia vapor after passing through the compressor may be less than 0.405 MPa, e.g., less than 0.404 MPa, less than 0.400 MPa, less than 0.390 MPa, less than 0.380 MPa, less than 0.370 MPa, less than 0.360 MPa, less than 0.350 MPa, less than 0.340 MPa, less than 0.330 MPa, less than 0.320 MPa, less than 0.310 MPa, or less than 0.300 MPa. In terms of lower limits, the final pressure of the ammonia vapor after passing through the compressor may be greater than 0.201 MPa, e.g., greater than 0.202 MPa, greater than 0.205 MPa, greater than 0.210 MPa, greater than 0.220 MPa, greater than 0.230 MPa, greater than 0.240 MPa, greater than 0.250 MPa, greater than 260 MPa, greater than 270 MPa, greater than 280 MPa, or greater than 0.290 MPa.

In some cases, the power demand is decreased, e.g., at least a 50% decrease, based on conventional operation. Operating the quench column at higher pressures, e.g., as disclosed herein, leads to a compressor power requirement decrease of over 80% in comparison to lower pressure operation, e.g., 0.013 MPa.

The temperature at the top of the quench column can range from 60° C. to 90° C., e.g., from 65° C. to 85° C., from 70° C. to 80° C., or from 75° C. to 80° C. In terms of upper limits, the temperature at the top of the quench column can be less than 90° C., e.g., less than 89° C., less than 88° C., less than 87° C., less than 86° C., less than 85° C., less than 84° C., less than 83° C., less than 82° C., less than 81° C., less than 80° C., less than 79° C., less than 78° C., less than 77° C., less than 76° C., or less than 75° C. In terms of lower limits, the temperature at the top of the quench column can be greater than 60° C., e.g., greater than 61° C., greater than 62° C., greater than 63° C., greater than 64° C., greater than 65° C., greater than 66° C., greater than 67° C., greater than 68° C., greater than 69° C., greater than 70° C., greater than 71° C., greater than 72° C., greater than 73° C., or greater than 74° C. At higher temperatures, product losses may increase due to increased ACN in the wastewater.

The temperature at the bottom of the quench column can range from 160° C. to 180° C., e.g., from 161° C. to 179° C., from 162° C. to 178° C., from 163° C. to 177° C., from 164° C. to 176° C., from 165° C. to 175° C., from 166° C. to 174° C., from 167° C. to 173° C., from 168° C. to 172° C., or from 169° C. to 171° C. In terms of upper limits, the temperature at the bottom of the quench column can be less than 180° C., e.g., less than 179° C., less than 178° C., less than 177° C., less than 176° C., less than 175° C., less than 174° C., less than 173° C., less than 172° C., less than 171° C., or less than 170° C. In terms of lower limits, the temperature at the bottom of the quench column can be greater than 160° C., e.g., greater than 161° C., greater than 162° C., greater than 163° C., greater than 164° C., greater than 165° C., greater than 166° C., greater than 167° C., greater than 168° C., or greater than 169° C. At higher temperatures, the ACN product may be lost in the aqueous phase of the liquid ammonia recovery stream (discussed below). At lower temperatures, water may be undesirably included in the ACN product.

### Separation Stream Compositions

In some cases, the vapor ammonia recovery stream comprises (higher amounts of) ammonia and water. The vapor recovery stream may comprise ammonia in an amount ranging from 90 wt. % to 99.9 wt. %, e.g., from 91 wt. % to 99 wt. %, from 92 wt. % to 98 wt. %, from 93 wt. % to 97 wt. %, or from 94 wt. % to 96 wt. %. In terms of lower limits, the amount of ammonia in the vapor recovery stream may be greater than 90 wt. % ammonia, e.g., greater than 90 wt. %, greater than 91 wt. %, greater than 92 wt. %, greater than 93 wt. %, greater than 94 wt. % ammonia, or greater than 95 wt. % ammonia. In terms of upper limits, the amount of ammonia in the vapor recovery stream may be less than 99.9 wt. % ammonia, e.g., less than 99 wt. % ammonia, less than 98 wt. % ammonia, less than 97 wt. % ammonia, or less than 96 wt. % ammonia.

In some cases, the vapor recovery stream may comprise water in an amount ranging from 0.1 wt. % to 10 wt. %, e.g., from 1 wt. % to 9 wt. %, from 2 wt. % to 8 wt. %, from 3 wt. % to 7 wt. %, or from 4 wt. % to 6 wt. %. In terms of lower limits, the amount of water in the vapor recovery stream may be greater than 0.1 wt. %, e.g., greater than 1 wt. %, greater than 2 wt. %, greater than 3 wt. %, or greater than 4 wt. %. In terms of upper limits, the amount of water in the vapor recovery stream may be less than 10 wt. %, e.g., less than 9 wt. %, less than 8 wt. %, less than 7 wt. %, less than 6 wt. %, or less than 5 wt. %.

The liquid recovery stream may comprise ammonia in an amount ranging from 10 wt. % to 40 wt. %, e.g., from 15 wt. % to 35 wt. %, or from 20 wt. % to 30 wt. %. In terms of lower limits, the liquid recovery stream may comprise ammonia in an amount greater than 10 wt. %, e.g., greater than 11 wt. %, greater than 12 wt. %, greater than 13 wt. %, greater than 14 wt. %, greater than 15 wt. %, greater than 16 wt. %, greater than 17 wt. %, greater than 18 wt. %, greater than 19 wt. %, greater than 20 wt. %, greater than 21 wt. %, greater than 22 wt. %, greater than 23 wt. %, or greater than 24 wt. %. In terms of upper limits, the liquid recovery stream may comprise ammonia in an amount less than 40 wt. %, e.g., less than 39 wt. %, less than 38 wt. % less than 37 wt. %, less than 36 wt. %, less than 35 wt. %, less than 34 wt. %, less than 33 wt. %, less than 32 wt. %, less than 31 wt. %, less than 30 wt. %, less than 29 wt. %, less than 28 wt. %, less than 27 wt. %, less than 26 wt. %, or less than 25 wt. %.

The liquid recovery stream may comprise water in an amount ranging from 50 wt. % to 80 wt. %, e.g., from 55 wt. % to 75 wt. %, or from 60 wt. % to 70 wt. %. In terms of upper limits, the liquid recovery stream may comprise water in an amount less than 80 wt. %, e.g., less than 79 wt. %, less than 78 wt. %, less than 77 wt. %, less than 76 wt. %, less than 75 wt. %, less than 74 wt. %, less than 73 wt. %, less than 72 wt. %, less than 71 wt. %, less than 70 wt. %, less than 69 wt. %, less than 68 wt. %, less than 67 wt. %, less than 66 wt. %, or less than 65 wt. %. In terms of lower limits, the liquid recovery stream may comprise water in an amount greater than 50 wt. %, greater than 51 wt. %, greater than 52 wt. %, greater than 53 wt. %, greater than 54 wt. %, greater than 55 wt. %, greater than 56 wt. %, greater than 57 wt. %, greater than 58 wt. %, greater than 59 wt. %, greater than 60 wt. %, greater than 61 wt. %, greater than 62 wt. %, greater than 63 wt. %, or greater than 64 wt. %.

The liquid recovery stream may further include its impurities, such as cyclohexanone, cyclohexenone, n-hexanenitrile, or 5-hexenenitrile, or combinations thereof. The liquid recovery stream may comprise its impurities in an amount ranging from 1 ppm to 5 wt. %, e.g., from 5 ppm to 4 wt. %, from 10 ppm to 3 wt. %, from 50 ppm to 2 wt. %, from 100 ppm to 1 wt. %, from 200 ppm to 9000 ppm, from 500 ppm to 8000 ppm, from 1000 ppm to 7000 ppm, from 1500 ppm to 6000 ppm, from 2000 ppm to 5000 ppm, or from 2500 ppm to 4500 ppm, from 3000 ppm to 4000 ppm. In terms of upper limits, the liquid recovery stream may comprise its impurities in an amount less than 5 wt. %, e.g., less than 4 wt. %, less than 3 wt. %, less than 2 wt. %, less than 1 wt. %, less than 9000 ppm, less than 8000 ppm, less than 7000 ppm, less than 6000 ppm, less than 5000 ppm, less than 4500 ppm, or less than 4000 ppm. In terms of lower limits, the liquid recovery stream may comprise its impurities in an amount greater than 1 ppm, greater than 5 ppm, greater than 10 ppm, greater than 50 ppm, greater than 100 ppm, greater than 200 ppm, greater than 500 ppm, greater than 1000 ppm, greater than 1500 ppm, greater than 2000 ppm, greater than 2500 ppm, or greater than 3000 ppm.

In some embodiments, the intermediate ACN stream comprises ACN along with impurities, e.g., high boiling impurities. These impurities may comprise aminocaproic acid, caprolactam, caprolactam dimers, caprolactam oligomers, or N-phenylacetamides, or combinations thereof.

The amount of ACN in the intermediate product stream may range from 20 wt. % to 60 wt. %, e.g., from 22 wt. % to 58 wt. %, from 24 wt. % to 56 wt. %, from 26 wt. % to 54 wt. %, from 28 wt. % to 52 wt. %, from 26 wt. % to 50 wt. %, from 28 wt. % to 48 wt. %, from 30 wt. % to 46 wt. %, from 32 wt. % to 44 wt. %, for 34 wt. % to 42 wt. %, or from 36 wt. % to 40 wt. %. In terms of lower limits, the intermediate stream may comprise ACN in an amount greater than 20 wt. %, e.g., greater than 22 wt. %, greater than 24 wt. %, greater than 26 wt. %, greater than 28 wt. %, greater than 30 wt. %, greater than 32 wt. %, greater than 34 wt. %, greater than 36 wt. %, greater than 38 wt. %, or greater than 40 wt. %. In terms of upper limits, the intermediate stream may comprise ACN in an amount less than 60 wt. %, e.g., less than 58 wt. %, less than 56 wt. %, less than 54 wt. %, less than 52 wt. %, less than 50 wt. %, less than 48 wt. %, less than 46 wt. %, less than 44 wt. %, or less than 42 wt. %.

The intermediate stream may further include caprolactam, as well as high boiling impurities such as aminocaproic acid, caprolactam dimers, caprolactam oligomers, N-phenylacetamides, or combinations thereof.

The intermediate stream may comprise caprolactam in an amount of 20 wt. % to 60 wt. %, e.g., from 22 wt. % to 58 wt. %, from 24 wt. % to 56 wt. %, from 26 wt. % to 54 wt. %, from 28 wt. % to 52 wt. %, from 26 wt. % to 50 wt. %, from 28 wt. % to 48 wt. %, from 30 wt. % to 46 wt. %, from 32 wt. % to 44 wt. %, for 34 wt. % to 42 wt. %, or from 36 wt. % to 40 wt. %. In terms of lower limits, the intermediate stream may comprise caprolactam in an amount greater than 20 wt. %, e.g., greater than 22 wt. %, greater than 24 wt. %, greater than 26 wt. %, greater than 28 wt. %, greater than 30 wt. %, greater than 32 wt. %, greater than 34 wt. %, greater than 36 wt. %, greater than 38 wt. %, or greater than 40 wt. %. In terms of upper limits, the intermediate stream may comprise caprolactam in an amount less than 60 wt. %, e.g., less than 58 wt. %, less than 56 wt. %, less than 54 wt. %, less than 52 wt. %, less than 50 wt. %, less than 48 wt. %, less than 46 wt. %, less than 44 wt. %, or less than 42 wt. %.

The intermediate stream may comprise high boiling impurities in an amount ranging from 1 wt. % to 10 wt. %, e.g., from 2 wt. % to 9 wt. %, from 3 wt. % to 8 wt. %, from 4 wt. % to 7 wt. %, or from 5 wt. % to 6 wt. %. In terms of upper limits, the intermediate stream may comprise high boiling impurities in an amount less than 10 wt. %, e.g., less than 9 wt. %, less than 8 wt. %, less than 7 wt. %, less than 6 wt. %. In terms of lower limits, the intermediate stream may comprise high boiling impurities in an amount greater than 1 wt. %, e.g., greater than 2 wt. %, greater than 3 wt. %, greater than 4 wt. %, or greater than 5 wt. %.

### Compression Step

As discussed above, the process may further comprise the step of compressing the ammonia recovery stream to form a compressed ammonia recovery stream. The compressing step increases the pressure of the recycled ammonia to better facilitate re-introduction to the reactor. The compression step may advantageously operate at low energy requirements because the pressure of the initial ammonia recovery stream is higher due to the higher pressure operation of the quench column. In contrast, conventional processes operate at lower P and detrimentally require a much higher degree of compression.

The type of compressor may vary widely. For example, the compressor may be a centrifugal compressor or a reciprocating compressor. The centrifugal compressor has been found to be advantageous because a low compression ratio may be achieved.

As noted above, unreacted ammonia may remain in the ammonia recovery stream(s). In some cases, the process advantageously recycles the unreacted ammonia back into the reaction to promote reaction efficiency and avoid wasting resources. The recycling may be achieved by passing the ammonia recovery stream(s) through the aforementioned compression step prior to its use in the reaction, as noted above, then conveying the compressed vapor ammonia recovery stream to the ammonia feed and/or directly to the reactor. The higher pressure separation/quench of the present disclosure decrease power demand on the compressor, thus increasing its lifespan and thereby lowering operating costs. In contrast, conventional low pressure quenching places greater strain on the ammonia compressor.

### Additional Ammonia Recovery

In some embodiments, the process further comprises the step of separating the liquid ammonia recovery stream to produce a supplemental ammonia recovery stream comprising ammonia and a bottoms stream comprising mostly water. As was the case with the first and second ammonia recovery streams, the process may also comprise the step of recycling at least a portion of the supplemental ammonia recovery stream (along with the first and/or second ammonia recovery stream) to the ammonia feed stream or to the contacting step.

In some embodiments, the supplemental ammonia recovery stream comprises ammonia in an amount ranging from 90 wt. % to 100 wt. %, e.g., from 91 wt. % to 99 wt. %, from 92 wt. % to 98 wt. %, from 93 wt. % to 97 wt. %, or from 94 wt. % to 96 wt. %. In terms of upper limits, the amount of ammonia in the overhead stream may be less than 100 wt. %, e.g., less than 99 wt. %, less than 98 wt. %, less than 97 wt. %, less than 96 wt. %, or less than 95 wt. %. In terms of lower limits, the amount of ammonia in the overhead stream may be greater than 90 wt. %, greater than 91 wt. %, greater than 92 wt. %, greater than 93 wt. %, or greater than 94 wt. %.

In some embodiments, the supplemental ammonia recovery stream comprises water in an amount ranging from 0 wt. % to 20 wt. %, e.g., from 1 wt. % to 19 wt. %, from 2 wt. % to 18 wt. %, from 3 wt. % to 18 wt. %, from 4 wt. % to 17 wt. %, from 5 wt. % to 16 wt. %, from 6 wt. % to 15 wt. %, from 7 wt. % to 14 wt. %, from 8 wt. % to 13 wt. %, from 9 wt. % to 12 wt. %, or from 10 wt. % to 11 wt. %. In terms of upper limits, the supplemental ammonia recovery stream may comprise water in an amount less than 20 wt. %, e.g., less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, or less than 10 wt. %. In term of lower limits, the supplemental ammonia recovery stream may comprise water in an amount greater than 0 wt. %, e.g., greater than 1 wt. %, greater than 2 wt. %, greater than 3 wt. %, greater than 4 wt. %, greater than 5 wt. %, greater than 6 wt. %, greater than 7 wt. %, greater than 8 wt. %, or greater than 9 wt. %.

In some cases, the bottoms stream comprises water in an amount ranging from 95 wt. % to 100 wt. %, e.g., from 96 wt. % to 99 wt. %, or from 97 wt. % to 98 wt. %. In terms of upper limits, the bottoms stream may comprise water in an amount greater than 95 wt. %, e.g., greater than 96 wt. %, or greater than 97 wt. %. In terms of lower limits, the bottoms stream may comprise water in an amount less than 100 wt. %, e.g., less than 99 wt. % or less than 98 wt. %.

The bottoms stream may further comprise ammonia in an amount ranging from 0 wt. % to 5 wt. %, e.g., from 1 wt. % to 4 wt. % or from 2 wt. % to 3 wt. %. In terms of upper limits, the bottoms stream may comprise ammonia in an amount less than 5 wt. %, e.g., less than 4 wt. % or less than 3 wt. %. In terms of lower limits, the bottoms stream may comprise ammonia in an amount greater than 0 wt. %, e.g., greater than 1 wt. % or greater than 2 wt. %.

The bottoms stream may further comprise impurities such as cyclohexanone, cyclohexenone, n-hexanenitrile, 5-hexenenitrile, for example. The bottoms stream may comprise the low-boiling impurities in an amount ranging from 0 wt. % to 5 wt. %, e.g., from 1 wt. % to 4 wt. % or from 2 wt. % to 3 wt. %. In terms of upper limits, the bottoms stream may comprise impurities in an amount less than 5 wt. %, e.g., less than 4 wt. % or less than 3 wt. %. In terms of lower limits, the bottoms stream may comprise impurities in an amount greater than 0 wt. %, e.g., greater than 1 wt. % or greater than 2 wt. %.

### Purification of Intermediate ACN Streams

### Crude Column

The intermediate ACN product stream(s) may be conveyed to a (crude) distillation column. The purification may comprise the step of separating the intermediate ACN product stream to produce a first purified product stream and a crude column bottoms.

The first purified product stream may comprise ACN in an amount ranging from 90 wt. % to 99 wt. %, e.g., from 91 wt. % to 98 wt. %, from 92 wt. % to 97 wt. %, from 93 wt. % to 96 wt. %, or from 94 wt. % to 96 wt. %. In terms of upper limits, the amount of ACN present in the first purified product stream may be less than 99 wt. %, e.g., less than 98 wt. %, less than 97 wt. %, less than 96 wt. %, or less than 95 wt. %. In terms of lower limits, the amount of ACN present in the first purified product stream may be greater than 90 wt. %, e.g., greater than 91 wt. %, greater than 92 wt. %, greater than 93 wt. %, greater than 95 wt. %, greater than 97 wt %, or greater than 99 wt %.

The first purified product stream may further comprise impurities such as cyclohexanone, cyclohexenone, n-hexanenitrile, 5-hexenitrile, aminocaproic acid, caprolactam, caprolactam dimers caprolactam oligomers, n-phenylacetamide, or combinations thereof.

The total amount of impurities in the first purified product stream may range from 1 wt. % to 9 wt. %, e.g., from 2 wt. % to 8 wt. %, from 3 wt. % to 7 wt. %, or from 4 wt. % to 6 wt. %. In terms of upper limits, the total amount of impurities in the first product stream may be less than 9 wt. %, e.g., less than 8 wt. %, less than 7 wt. %, less than 6 wt. %, or less than 5 wt. %. In terms of lower limits, the total amount of impurities in the first purified product stream may be greater than 1 wt. %, e.g., greater than 2 wt. %, greater than 3 wt. %, greater than 4 wt. %, or greater than 5 wt. %.

In some cases, the crude column bottoms comprises caprolactam in an amount ranging from 80 wt. % to 99 wt. %, e.g., from 81 wt. % to 98 wt. %, from 82 wt. % to 97 wt. %, from 83 wt. % to 96 wt. %, from 84 wt. % to 95 wt. %, from 85 wt. % to 94 wt. %, from 86 wt. % to 93 wt. %, from 87 wt. % to 92 wt. %, from 88 wt. % to 91 wt. %, or from 89 wt. % to 90 wt. %. In terms of upper limits, the crude column bottoms may comprise caprolactam in an amount less than 99 wt. %, e.g., less than 98 wt. %, less than 97 wt. %, less than 96 wt. %, less than 95 wt. %, less than 94 wt. %, less than 93 wt. %, less than 92 wt. %, less than 91 wt. %, or less than 90 wt. %. In terms of lower limits, the crude column bottoms may comprise caprolactam in an amount greater than 80 wt. %, e.g., greater than 81 wt. %, greater than 82 wt. %, greater than 83 wt. %, greater than 84 wt. %, greater than 85 wt. %, greater than 86 wt. %, greater than 87 wt. %, greater than 88 wt. %, or greater than 90 wt. %.

### Capro Column

The purification may further comprise the step of separating the crude column bottoms to produce a distillate caprolactam recycle stream comprising greater than 95 wt % caprolactam and heavy byproducts stream containing organic impurities and less than 10 wt % caprolactam. In some cases, the purification further comprises the step of recycling the caprolactam recycle stream to the caprolactam feed stream or to the reaction step.

The amount of caprolactam in the caprolactam recycle stream may range from 90 wt. % to 100 wt. %, e.g., from 91 wt. % to 99 wt. %, from 92 wt. % to 98 wt. %, from 93 wt. % to 97 wt. %, or from 94 wt. % to 96 wt. %. In terms of lower limits, the amount of caprolactam in the caprolactam recycle stream may be greater than 90 wt. %, e.g., greater than 91 wt. %, greater than 92 wt. %, greater than 93 wt. %, greater than 94 wt. %, or greater than 95 wt. %. In terms of upper limits, the amount of caprolactam in the caprolactam recycle stream may be less than 100 wt. %, e.g., less than 99 wt. %, less than 98 wt. %, less than 97 wt. %, or less than 96 wt. %.

The caprolactam recycle stream may be recycled back to the reaction in the caprolactam feed stream.

### Product Column

The purification may further comprise the step of separating the first purified product stream to form a second purified product stream comprising highly pure ACN, e.g., greater than 95 wt % ACN or greater than 99 wt % ACN. The separation may also produces a product column bottoms stream comprising mostly heavies similar to those of the heavy byproducts stream.

The amount of ACN in the second purified product stream may range from 90 wt. % to 100 wt. %, e.g., from 91 wt. % to 99 wt. %, from 92 wt. % to 98 wt. %, from 93 wt. % to 97 wt. %, or from 94 wt. % to 96 wt. %. In terms of lower limits, the amount of ACN in the purified product stream may be greater than 90 wt. %, e.g., greater than 91 wt. %, greater than 92 wt. %, greater than 93 wt. %, greater than 94 wt. %, or greater than 95 wt. %. In terms of upper limits, the amount of ACN in the purified product stream may be less than 100 wt. %, e.g., less than 99 wt. %, less than 98 wt. %, less than 97 wt. %, or less than 96 wt. %.

The disclosed process may be conducted as a continuous process, although a batch process is also contemplated. The continuous process provides for production benefits. As discussed above, the continuous process allows for a lower residence times in the various distillation columns, e.g., quench column and/or wastewater column. Such short residence times are not typically achieved when processes are conducted in a batch manner, e.g., via batch distillation. Without wishing to be bound by theory, in some cases, continuous processes and short residence times work synergistically, to limit undesired side reactions of the ACN product and resulting in higher recovery of the ACN product. For example, during longer residence times, ACN may react with caprolactam, or oligomers thereof thereby siphoning off some of the desired ACN product. The products of these undesired side reactions may include, for example, N-(5-cyanopentyl)-aminocaproamide. ACN may undergo thermal degradation during longer residence times to product undesired side products, such as bis-(5-cyanopentyl)-amine, for example. ACN may also undergo hydrolysis reactions during longer residence times. Products of ACN hydrolysis may include, for example, aminocaproic acid, and aminocaproamide.

Further, longer residence times may result in oligomerization/polymerization of ACN and reactions of its oligomer or polymers with caprolactam or caproic acid, for example. Caprolactam may also undergo oligomerization/polymerization during longer residence times, thereby limiting the amount of caprolactam available for recycling.

In some cases, residence times may range from 5 minutes to 180 minutes, e.g., from 5 minutes to 60 minutes, from 10 minutes to 170 minutes, from 15 minutes to 160 minutes, from 20 minutes to 150 minutes, from 25 minutes to 140 minutes, from 30 minutes to 120 minutes, from 40 minutes to 110 minutes, from 50 minutes to 100 minutes, from 60 minutes to 90 minutes, or from 70 minutes to 80 minutes. In terms of upper limits, the residence times may be less than 180 minutes, e.g., less than 170 minutes, less 160 minutes, less than 150 minutes, less than 140 minutes, less than 130 minutes, less than 120 minutes, less than 110 minutes, less than 100 minutes, less than 90 minutes, less than 80 minutes, or less than 70 minutes. In terms of lower limits, the residence times may be greater than 5 minutes, e.g., greater than 10 minutes, greater than 15 minutes, greater than 20 minutes, greater than 25 minutes, greater than 30 minutes, greater than 35 minutes, greater than 40 minutes, greater than 45 minutes, greater than 50 minutes, greater than 55 minutes, greater than 60 minutes, or greater than 65 minutes. Lower residence times may result in incomplete separation. Higher residence times may result in lowered yield. These ranges and limits are applicable to all units of the disclosed process.

In some embodiments, the disclosed process may be conducted as a large-scale continuous process operated at a throughput rate ranging from 50 million to 500 million pounds per year; e.g., from 55 million to 450 million, from 60 million to 400 million, from 70 million to 350 million, from 80 million to 300 million, or from 90 million to 250, or from 100 million to 200 million. In terms of lower limits, the throughput rate may be greater than 50 million pounds per year, e.g., greater than 60 million, greater than 70 million, greater than 80 million, greater than 90 million, or greater than 100 million. In terms of upper limits, the throughput rate may be less than 500 million pounds per year, e.g., less than 450 million, less than 400 million, less than 350 million, less than 300 million, less than 250 million, or less than 200 million. This additional benefit of operating the process in a continuous manner provides for increases in overall production.

### Continuous Distillation of Crude ACN

Alternatively, the crude ACN may be subjected to purification via a continuous process to produce an intermediate ACN stream and a caprolactam recovery stream. In some cases, the continuous process may comprise distillation in one or more columns. The continuous distillation may be conducted without a separate quench column. The intermediate ACN stream may be further purified to produce a purified product stream.

Advantageously, the continuous process has been shown to limit undesired side reactions. Without being bound by theory, it is postulated that shorter residence time in any one column limits or precludes further reactions of ACN and/or caprolactam. Recovery of both ACN and caprolactam are therefore improved when using the continuous process described herein in comparison to conventional batch distillation processes.

In these cases, the temperature at the bottom of the distillation column can range from 150° C. to 180° C., e.g., from 151° C. to 179° C., from 152° C. to 178° C., from 153° C. to 177° C., from 154° C. to 176° C., from 155° C. to 175° C., from 156° C. to 174° C., from 157° C. to 173° C., from 158° C. to 172° C., from 159° C. to 171° C., from 160° C. to 170° C., from 161° C. to 169° C., from 162° C. to 168° C., from 163° C. to 167° C., or from 164° C. to 166° C. In terms of upper limits, the temperature at the bottom of the distillation column can be less than 180° C., e.g., less than 179° C., less than 178° C., less than 177° C., less than 176° C., less than 175° C., less than 174° C., less than 173° C., less than 172° C., less than 171° C., less than 170° C., less than 169° C., less than 168° C., less than 167° C., less than 166° C., or less than 165° C. In terms of lower limits, the temperature at the bottom of the distillation column can be greater than 150° C., e.g., greater than 151° C., greater than 152° C., greater than 153° C., greater than 154° C., greater than 155° C., greater than 156° C., greater than 157° C., greater than 158° C., greater than 159° C., greater than 160° C., greater than 161° C., greater than 162° C., greater than 163° C., or greater than 164° C.

The pressure may range from 0.1 kPa to 100 kPa, e.g., from 0.5 kPa to 99 kPa, from 1 kPa to 95 kPa, from 2 kPa to 85 kPa, from 10 kPa to 80 kPa, from 15 kPa to 75 kPa, from 20 kPa to 70 kPa, from 25 kPa to 65 kPa, from 30 kPa to 60 kPa, from 35 kPa to 55 kPa, or from 40 kPa to 50 kPa. In terms of upper limits, the pressure may be less than 100 kPa, e.g., less than 95 kPa, less than 90 kPa, less than 85 kPa, less than 80 kPa, less than 75 kPa, less than 70 kPa, less than 65 kPa less than 60 kPa, less than 55 kPa, less than 50 kPa, or less than 45 kPa. In terms of lower limits, the pressure may be greater than 0.1 kPa, e.g., greater than 0.5 kPa, greater than 1 kPa, greater than 2 kPa, greater than 5 kPa, greater than 10 kPa, greater than 15 kPa, greater than 20 kPa, greater than 25 kPa, greater than 30 kPa, greater than 35 kPa, or greater than 40 kPa.

Residence times in any one column may range from 5 minutes to 450 minutes, e.g., from 5 minutes to 400 minutes, from 10 minutes to 350 minutes, from 15 minutes to 300 minutes, from 20 minutes to 250 minutes, from 25 minutes to 200 minutes, from 30 minutes to 180 minutes, from 40 minutes to 150 minutes, from 50 minutes to 120 minutes, from 60 minutes to 90 minutes, or from 70 minutes to 80 minutes. In terms of upper limits, the residence times may be less than 450 minutes, e.g., less than 400 minutes, less than 350 minutes, less than 300 minutes, less than 250 minutes, less than 200 minutes, less than 180 minutes, e.g., less than 170 minutes, less 160 minutes, less than 150 minutes, less than 140 minutes, less than 130 minutes, less than 120 minutes, less than 110 minutes, less than 100 minutes, less than 90 minutes, less than 80 minutes, or less than 70 minutes. In terms of lower limits, the residence times may be greater than 5 minutes, e.g., greater than 10 minutes, greater than 15 minutes, greater than 20 minutes, greater than 25 minutes, greater than 30 minutes, greater than 35 minutes, greater than 40 minutes, greater than 45 minutes, greater than 50 minutes, greater than 55 minutes, greater than 60 minutes, or greater than 65 minutes. Lower residence times may result in incomplete separation. Higher residence times may result in lowered yield.

It has surprisingly been found that the reflux ratio may unexpectedly contribute to superior performance. Without wishing to be bound by theory, it is thought that increasing the reflux ratio may reduce the amount of caprolactam in the distillate (intermediate product) stream.

The reflux ratio during distillation may range from 1:1 to 10:1, e.g., from 1.5:1 to 9.5:1, from 2:1 to 9:1, from 2.5:1 to 8.5:1, from 3:1 to 8:1, from 3.5:1 to 7.5:1, from 4:1 to 7:1, from 4.5:1 to 6.5:1, or from 5:1 to 6:1. In some embodiments, the reflux ratio may be 2.5:1+/−10%. In some embodiments, the reflux ratio may be 5:1+/−10%.

It has surprisingly been found that the feed rate may also contribute to performance. Without wishing to be bound by theory, it is postulated that a higher feed rate lessens the residence time in the column sump, thereby reducing undesirable side reactions.

In some cases, the feed rate may range from 1 mL/min to 10 mL/min, e.g., from 1.5 mL/min to 9.5 mL/min, from 2 mL/min to 9 mL/min, from 2.5 mL/min to 8.5 mL/min, from 3 mL/min to 8 mL/min, from 3.5 mL/min to 7.5 mL/min, from 4 mL/min to 7 mL/min, from 4.5 mL/min to 6.5 mL/min, or from 5 mL/min to 6 mL/min. In terms of upper limits, the feed rate may be less than 10 mL/min, e.g., less than 9.5 mL/min, less than 9 mL/min, less than 8.5 mL/min, less than 8 mL/min, less than 7.5 mL/min, less than 7 mL/min, less than 6.5 mL/min, less than 6 mL/min, or less than 5.5 mL/min. In terms of lower limits, the feed rate may be greater than 1 mL/min, e.g., greater than 1.5 mL/min, greater than 2 mL/min, greater than 2.5 mL/min, greater than 3 mL/min, greater than 3.5 mL/min, greater than 4 mL/min, greater than 4.5 mL/min, or greater than 5 mL/min.

In some cases, e.g., on larger scale (100-600 MLb/year), the feed rate may range from 1 klb/hr to 250 klb/hr, e.g., from 5 klb/hr to 240 klb/hr, from 10 klb/hr to 230 klb/hr, from 20 klb/hr to 220 klb/hr, from 30 klb/hr to 210 klb/hr, from 40 klb/hr to 200 klb/hr, from 50 klb/hr to 190 klb/hr, from 60 klb/hr to 180 klb/hr, from 70 klb/hr to 170 klb/hr, from 80 klb/hr to 160 klb/hr, from 90 klb/hr to 150 klb/hr, from 100 klb/hr to 140 klb/hr, from 110 klb/hr to 130 klb/hr, or from 115 klb/hr to 125 klb/hr. In terms of upper limits, the feed rate may be less than 200 klb/hr, e.g., less than 190 klb/hr, less than 180 klb/hr, less than 170 klb/hr, less than 160 klb/hr, less than 150 klb/hr, less than 140 klb/hr, less than 130 klb/hr, less than 125 klb/hr, or less than 120 klb/hr. In terms of lower limits, the feed rate may be greater than 1 klb/hr, e.g., greater than 5 klb/hr, greater than 10 klb/hr, greater than 20 klb/hr, greater than 30 klb/hr, greater than 40 klb/hr, greater than 50 klb/hr, greater than 60 klb/hr, greater than 70 klb/hr, greater than 80 klb/hr, greater than 90 klb/hr, greater than 100 klb/hr, greater than 110 klb/hr, or greater than 115 klb/hr.

In some cases, ACN recovery in the intermediate ACN stream may be greater than 70%, e.g., greater than 75%, greater than 80%, greater than 85%, greater than 90%, greater than 91%, greater than 92%, greater than 93%, greater than 94%, greater than 95%, greater than 96%, greater than 97%, greater than 98%, greater than 99%, greater than 99.5%, or greater than 99.9%. In terms of upper limits, ACN recovery may be less than 100%, e.g., less than 99.9%, less than 99.5%, less than 99%, less than 98%, less than 97%, or less than 96%. In terms of lower limits, ACN recovery may be greater than 70%, e.g., greater than 75%, greater than 85%, greater than 90%, greater than 91%, greater than 92%, greater than 93%, greater than 94%, or greater than 95%.

In some cases, caprolactam recovery in the caprolactam recovery stream may be greater than 90%, e.g., greater than 91%, greater than 92%, greater than 93%, greater than 94%, greater than 95%, greater than 96%, greater than 97%, greater than 98%, greater than 99%, greater than 99.5%, or greater than 99.9%. In terms of upper limits, caprolactam recovery may be less than 100%, e.g., less than 99.9%, less than 99.5%, less than 99%, less than 98%, less than 97%, or less than 96%. In terms of lower limits, caprolactam recovery may be greater than 90%, e.g., greater than 91%, greater than 92%, greater than 93%, greater than 94%, or greater than 95%.

### Purification of Intermediate ACN Stream

The intermediate ACN stream may be further purified, for example, via distillation in one or more columns, to produce a purified product stream. Specifically, the intermediate ACN stream may be distilled to remove low-boiling point contaminants (lights). The low-boiling point contaminants may comprise cyclopentaneamine, pentanenitrile, cyclopentanol, cyclopentanone, 5-cyano-1-pentene, and hexamethyleneimine, for example, along with ACN, and a bottoms stream comprising the purified product.

The temperature at the bottom of the column may range from 110° C. to 150° C., e.g., from 115° C. to 145° C., from 120° C. to 140° C., or from 125° C. to 135° C. In terms of upper limits, the temperature at the bottom of the column may be less than 150° C., e.g., less than 145° C., less than 140° C., less than 135° C., or less than 130° C. In terms of lower limits, the temperature at the bottom of the column may be greater than 110° C., greater than 115° C., greater than 120° C., or greater than 125° C.

The pressures and reflux ratios during purification of the intermediate stream may independently fall within the ranges provided above for the continuous distillation of crude ACN.

The recovery of ACN in the purified product stream may be greater than 90%, e.g., greater than 91%, greater than 92%, greater than 93%, greater than 94%, greater than 95%, greater than 96%, greater than 97%, greater than 98%, greater than 99%, greater than 99.5%, or greater than 99.9%. In terms of upper limits, the recovery of ACN in the purified product stream may be less than 100%, e.g., less than 99.9%, less than 99.5%, less than 99%, less than 98%, less than 97%, or less than 96%. In terms of lower limits, the recovery of ACN in the purified products stream may be greater than 90%, e.g., greater than 91%, greater than 92%, greater than 93%, greater than 94%, or greater than 95%.

The purity of the ACN in the purified product stream may be greater than 95%, e.g., greater than 96%, greater than 97%, greater than 98%, greater than 99%, greater than 99.5%, greater than 99.9%, or greater than 99.99%. In terms of upper limits, the purity of the ACN in the purified product stream may be less than 100%, e.g., less than 99.99%, less than 99.9%, less than 99%, less than 98%, less than 97%, or less than 96%. In terms of lower limits, the purity of the ACN in the purified product stream may be greater than 90%, e.g., greater than 91%, greater than 92%, greater than 93%, greater than 94%, or greater than 95%.

### Lower Purity Caprolactam

In some embodiments, the caprolactam starting materials (the caprolactam feed stream) need not be of high purity, as is conventional. As noted above, this has been found to be beneficial because the formation of high purity streams often requires significant processing, which may be costly and complicated. When the disclosed process is employed, lower purity caprolactam feed may be used and similar, if not improved, reaction parameters are demonstrated.

In some cases, the low purity caprolactam feed comprises less than 100 wt. % caprolactam and impurities in amounts higher than those of conventional feed streams. In some cases, the impurities comprise aminocaproic acid, caprolactam dimers, caprolactam oligomers, N-methylcaprolactam, or N-ethylcaprolactam, N-methylvalerolactam, cyclohexanone, cyclohexenone, n-hexanenitrile, 5-hexenenitrile, or N-phenylacetamide, or combinations thereof.

The amount of impurities in the caprolactam feed stream can, for example, range from 100 ppm to 40 wt. %, e.g., 250 ppm to 35 wt. %, 500 ppm to 30 wt. %, 1000 ppm to 25 wt. %, 2500 ppm to 20 wt. %, 5000 ppm to 15 wt. %, or 1 wt. % to 10 wt. %. In terms of upper limits, the amount of impurities in the caprolactam feed stream can be less than 40 wt. %, e.g., less than 35 wt. %, less than 30 wt. %, less than 25 wt. %, less than 20 wt. %, or less than 15 wt. %. In terms of lower limits, the amount of impurities in the caprolactam feed stream can be greater than greater than 100 ppm, e.g., greater than 250 ppm, greater than 500 ppm, greater than 1000 ppm, greater than 2500 ppm, greater than 5000 ppm, or greater than 1 wt. %, or. Lower amounts, e.g., less than 100 ppm, and higher amounts, e.g., greater than 40 wt. %, are also contemplated. The ranges and limits mentioned herein are applicable to individual impurities and to impurities as a whole.

### Catalyst

The catalyst may advantageously comprise little, if any, of an active metal, e.g., less than 5 wt. % of an active metal, and/or little, if any, of other synergists, e.g., phosphorus. The use of the disclosed catalyst has been unexpectedly found to contribute to the aforementioned improvements in reaction efficiency.

The catalyst comprises a base catalyst and little, if any, active metal. In some cases, the base catalyst may comprise a zeolite catalyst. Many zeolite catalysts are known. For example, the base catalyst may comprise MFI (ZSM-5), FAU (faujasite, USY),*BEA (beta), MOR (high-silica mordenite), and FER (high-silica ferrierite).

The amount of active metals in the catalyst can range from 0 to 5 wt. %, e.g., from 1 ppb to 4 wt. %, from 10 ppb to 2 wt. %, from 100 ppb to 1 wt. %, from 1 ppm to 5000 ppm, from 10 ppm to 2500 ppm, from 50 ppm to 1000 ppm, from 100 ppm to 500 ppm, or from 150 ppm to 250 ppm. In terms of upper limits, the amount of active metals in the catalyst can be less than 5 wt. %, e.g., less than 4 wt. %, less than 2 wt. %, less than 1 wt. %, less than 5000 ppm, less than 2500 ppm, less than 1000 ppm, less than 500 ppm, less than 250 ppm, less than 100 ppm, less than 50 ppm, or less than 10 ppm. In terms of lower limits, the amount of active metals in the catalyst can be greater than 1 ppb, e.g., greater than 10 ppb, greater than 100 ppb, greater than 1 ppm, greater than 10 ppm, greater than 50 ppm, greater than 100 ppm, greater than 250 ppm, greater than 500 ppm, greater than 1000 ppm, or greater than 2500 ppm. Lower amounts, e.g., less than 1 ppb, and higher amounts, e.g., greater than 5 wt. %, are also contemplated.

In addition to comprising little, if any, active metals, the disclosed catalyst may comprise little, if any, synergists. One example of a synergist is phosphorus. The amount of phosphorous present in the catalyst can, for example, range from 0 to 1 wt. %, e.g., from 1 ppb to 5000 ppm, from 10 ppb to 2500 ppm, from 100 ppb to 1000 ppm, from 1 ppm to 500 ppm, from 50 ppm to 250 ppm, or from 100 ppm to 200 ppm. In terms of upper limits, the amount of phosphorous present in the catalyst can be less than 1 wt. %, e.g., less than 5000 ppm, less than 2500 ppm, less than 1000 ppm, or less than 500 ppm, less than 250 ppm, less than 100 ppm, less than 50 ppm, or less than 10 ppm. In terms of lower limits, the amount of phosphorous present in the catalyst can be greater than 1 ppb, e.g., greater than 10 ppb, greater than 100 ppb, greater than 1 ppm, greater than 10 ppm, greater than 25 ppm, greater, than 50 ppm, greater than 100 ppm, or greater than 250 ppm. Lower amounts, e.g., less than 1 ppb, and higher amounts, e.g., greater than 1 wt. %, are also contemplated.

The silica to alumina ratio (SAR) of the catalyst can, for example, range from 25 to 200, e.g., from 40 to 150, from 50 to 125, or from 75 to 100. In terms of upper limits, the SAR of the catalyst can be 200 or less, e.g., 150 or less, 125 or less, or 100 or less. In terms of lower limits, the SAR of the catalyst can be greater than 25, e.g., greater than 40, greater than 50, or greater than 75. Lower ratios, e.g., less than 25, and higher ratios, e.g., greater than 200, are also contemplated

The reaction may be conducted at a temperature ranging from 250° C. to 500° C., e.g., from 275° C. to 475° C., from 300° C. to 450° C., from 325° C. to 425° C., or from 350° C. to 400° C. In terms of upper limits, reaction may be conducted at a temperature less than 500° C., less than 475° C., less than 450° C., less than 425° C., or less than 400° C. In terms of lower limits, reaction may be conducted at a temperature greater than 250° C., greater than 275° C., greater than 300° C., greater than 350° C., or greater than 375° C. At temperatures below about 300° C., the reaction may not proceed. At temperatures greater than 450° C., the selectivity may decline.

The reaction may be conducted at a pressure ranging from 0 psig to 500 psig, e.g., 1 psig to 450 psig, 5 psig to 400 psig, 10 psig to 350 psig, 50 psig to 300 psig, 100 psig to 250 psig, or 150 psig to 200 psig. In terms of upper limits, the reaction pressure can be less than 500 psig, less than 450 psig, less than 400 psig, less than 350 psig, less than 300 psig, less than 250 psig, or less than 200 psig. In terms of lower limits, the reaction pressure can be greater than 1 psig, greater than 5 psig, greater than 10 psig, greater than 50 psig, greater than 100 psig, or greater than 150 psig. Lower pressures, e.g., less than 1 psig, and higher pressures, e.g. greater than 500 psig, are also contemplated.

The manner in which the reaction may be conducted may vary widely. In some cases, the reaction may be conducted in a fixed bed reactor. It has been discovered that fixed bed reactors surprisingly provide for improved uniformity of temperature and good contact between the catalyst and reactants. Advantageously, these reactors also contribute low equipment costs for construction, operation, and maintenance.

### Exemplary Separation Schemes

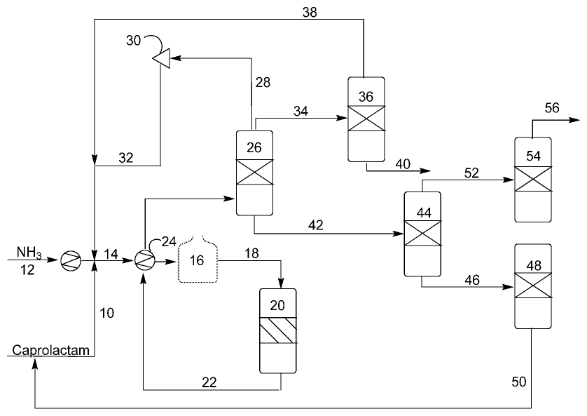

The present disclosure includes a process for converting caprolactam to ACN, e.g., 6-ACN. In some cases, the process may be a gas-phase process. In some cases, the process may be a liquid-phase process. As shown in FIG. 1, the process may comprise contacting a caprolactam feed stream 10 with an ammonia stream 12 to provide a combined stream 14. Combined stream 14 may then be passed to a preheater 16, prior to passing the preheated combined stream 18 to a reactor 20 comprising a catalyst (not shown) to produce a first crude product stream 22 comprising ACN. The crude product stream 22 may be passed through a cooler 24 before being passed to a quench column 26. From quench column 26, a vapor recovery stream 28 comprising ammonia may be passed to a compressor 30 to provide a first ammonia recovery stream 32 which may be recycled to combined steam 14. Quench column 26 may also provide a liquid recovery stream 34 comprising ammonia and water, which may be passed to a wastewater column 36 to provide a second ammonia recovery stream 38 comprising ammonia, which may be recycled back to combined stream 14, and a waste stream 40 comprising water. Quench column 26 further provides an intermediate stream 42 comprising ACN and higher boiling impurities. Intermediate stream 42 may be passed to a distillation column 44 to provide a caprolactam recovery stream 46, which may be passed to a caprolactam distillation column 48 to provide a purified caprolactam stream 50. Purified caprolactam stream 50 may be recycled back to caprolactam feed stream 10. Distillation column 44 further provides a product stream 52 comprising ACN. Product stream 52 may be passed to an ACN distillation column 54 to provide a high purity ACN stream 56, which may be stored or used in further reactions. The high purity ACN stream 56 may be removed as a side draw or an overhead product.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 4.0” may be interpreted as, and subsequently modified in the claims as “greater than or equal to 4.0.”

Some of the components and steps disclosed herein may be considered optional. In some cases, the disclosed compositions, processes, etc. may expressly exclude one or more of the aforementioned components or steps in this description, e.g., via claim language. This is contemplated herein by the inventors. For example, claim language may be modified to recite that the disclosed compositions, processes, streams, etc., do not utilize or comprise one or more of the aforementioned components or steps, e.g., e.g., the caprolactam feed stream does not include N-phenylacetamide (or any other of the aforementioned additives). Such negative limitations are contemplated, and this text serves as support for negative limitations for components, steps, and/or features.

### EXAMPLES

### Examples 1-26: Continuous Distillation of Crude ACN

A caprolactam feed stream (˜99.5% purity) comprising caprolactam, aminocaproic acid, hexenoic acid, caprolactam dimers, N-methylcaprolactam, and/or N-ethylcaprolactam was contacted with ammonia to produce a first crude product stream. The first crude product stream was preheated and charged to a separation system. The system comprised a distillation column including two packed sections separated by a feed section: a bottom section packed with 0.16 inch stainless steel packing, and a top section packed with 0.16 inch stainless steel packing. The feed section of the apparatus used adjustable pumps. The feed rate varied from 1.5 mL/min to 5 mL/min. Adjustable heating elements were used on the reboiler to adjust the base column temperature. The condenser water temperature was 30° C. and the pressure was 0.002 MPa. The reflux ratio was controlled by a magnetic reflux splitter on a timer.

The intermediate (distillate) stream was removed from a reflux splitter into the distillate receiver. The bottoms product was drawn via an overflow line on the column sump. Examples 1-22 are samples taken from the distillate and bottoms during the continuous run. The results were averaged, and the mass balance was calculated based on the bottoms and distillate flow rates and analytical results. The components were analyzed by gas chromatograph (GC), and the distillation conditions for the separation of the crude product stream as well as the separation results are shown below in Table 1.

TABLE 1

| | | | | Distillate | Bottoms | Distillate | Bottoms | Colum |
|---|---|---|---|---|---|---|---|---|
| | Feed rate | Reflux | | ACN | ACN | Capro. | Capro. | Bottom T |
| | mL/min | ratio | Water | wt. % | wt. % | wt. % | wt. % | ° C. |
| Avg. | 2 | 2.5 | 4.1 | 97.3 | 1.79 | 0.16 | 90.08 | 158.4 |

It was surprisingly found that a feed rate ranging from 1.5 mL/min to 5 mL/min unexpectedly contributed to superior performance, especially in combination with a reflux ratio of 2-3 and/or a column bottom temperature of approximately 159° C. to 161° C.

The averaged mass balance and recovery for both ACN and caprolactam are shown below in Table 2, along with the overall mass balance for the average values. Unexpectedly, the continuous distillation processes of Examples 1-22 demonstrate only small losses in ACN and caprolactam during the process. This is evidenced by the 99% mass balance for both components. Further, a high percent recovery of both components was observed. ACN recovery using the continuous distillation method of Examples 1-22 was significantly higher than the batch distillations of Comparative Examples A and B: 95% versus an average of 81%. See below for details of Comparative Examples A and B.

Without being bound by theory, it is postulated that less degradation of both ACN and caprolactam may occur during continuous distillation. It is thought that the shorter sump residence times in comparison to batch distillation results in fewer undesirable side reactions which lead to decreases in recovery of both ACN and caprolactam.

TABLE 2

| | Ex. 1-22 | Comp. Ex. A | Comp. Ex. B |
|---|---|---|---|
| ACN mass balance % | 99 | 106 | 85 |
| ACN recovery % | 95 | 86 | 76 |
| Capro. mass balance % | 99 | 83 | 88 |
| Capro. recovery % | 99 | 79 | 74 |
| Total mass balance | 97 | 88 | 94 |

Further, caprolactam recovery in the intermediate stream was significantly higher using the continuous distillation method in comparison to batch distillation—99% versus an average of 76.5% for Comparative Examples A and B.

### Example 23: Purification of the Intermediate Product Stream

An intermediate product stream as described above was subjected to further purification via continuous distillation to produce a purified product stream. The column bottom temperature was adjusted for the target ACN purity, specifically, a temperature at which water and low-boiling point contaminants (lights) could be removed. Once the targeted purity (greater than 99.5%) was reached, the reflux ratio was adjusted to maximize the recovery of ACN.

Seven samples were pulled from a run performed under the conditions shown below in Table 3. The samples were analyzed, and the averaged results and conditions are also shown below in Table 3. It was found that sampling over a longer duration under the aforementioned conditions ensured superior steady state and accurate mass balance. Specifically, each sample was approximately 200 mL collected over about 2 hours. Averaging multiple samples provided more accurate mass balance to due process variability during the collection of individual samples.

TABLE 3

| | | | | Distillate | Bottoms | Distillate | Bottoms | Colum |
|---|---|---|---|---|---|---|---|---|
| | Feed rate | Reflux | | ACN | ACN | lights | lights | Bottom T |
| | mL/min | ratio | Water | wt. % | wt. % | wt. % | wt. % | ° C. |
| Avg. | 2 | 5 | 1.8 | 72.2 | 98.8 | 21.1 | 0 | 124.7 |

The averaged mass balance and recovery for ACN is shown below in Table 4, along with the overall mass balance for the average values.

TABLE 4

| | |
|---|---|
| ACN mass balance % | 100 |
| ACN recovery % | 99.3 |
| Total mass balance | 98.9 |

As shown above, the recovery of ACN in the intermediate stream was significantly improved using the continuous distillation method described above: greater than 99% in comparison to an average of 77.5% in the batch distillation trials, see Comparative Examples A and B below in Table 5.

### Comparative Example A and B: Batch Distillation of Crude ACN

Comparative Examples A and B, referenced above, were conducted as follows. Similar caprolactam feed stream comprising caprolactam, aminocaproic acid, hexenoic acid, caprolactam dimers, N-methylcaprolactam, and/or N-ethylcaprolactam was contacted with ammonia to produce a first crude product stream.

The first crude product stream was distilled via batch distillation in a 2″ Oldershaw column with 30 trays. The distillation was conducted at higher pressures, e.g., greater than vacuum, as disclosed herein. Two separate runs were conducted. The reflux ratio was 10:1. In the first run, Comparative Ex. A, the pressure ranged from 0.001 MPa to 0.003 MPa. The column bottom temperature ranged from 167° C. to 178° C. and the column top temperature ranged from 104° C. to 137° C. In the second run, Comparative Ex. B, the pressure ranged from 0.001 MPa to 0.006 MPa. The column bottom temperature ranged from 106° C. to 175° C., and the column top temperature ranged from 40° C. to 119° C.

A total of seven cuts were collected and analyzed by GC from the run in Comp. Ex. A. A total of ten cuts were collected and analyzed by GC from the run in Comp. Ex. B. The results are shown below in Table 5.

TABLE 5

| | Mass Balance | | % Recovery | |
|---|---|---|---|---|
| | Comp. Ex. A | Comp. Ex. B | Comp. Ex. A | Comp. Ex. B |
| Water | 11% | 96% | | |
| Lights | 88% | 146% | | |
| ACN | 106% | 85% | 86% | 76% |
| Caprolactam | 83% | 88% | 79% | 74% |
| Heavies | 184% | 384% | | |
| Other | 54% | 580% | | |
| Total | 88% | 94% | | |

Mass balance following batch distillation suggests generation of heavy impurities during the process. Without wishing to be bound by theory, it is postulated that caprolactam may react with ACN in the pot (vessel in which the components are heated), generating undesired byproducts. This may be demonstrated by the mass balance values shown below in Table 5. Specifically, the apparent mass balance of greater than 100% for heavy impurities and less than 100% for desired components suggest that the desired components undergo undesirable side reactions in the pot to generate heavy impurities.

### EMBODIMENTS

As used below, any reference to a series of embodiments is to be understood as a reference to each of those embodiments disjunctively (e.g., “Embodiments 1-4” is to be understood as “Embodiments 1, 2, 3, or 4”).

Embodiment 1: a process for converting caprolactam to aminocapronitrile (ACN), the process comprising contacting a caprolactam feed stream with ammonia to produce a first crude product stream, separating the first crude product stream at a pressure greater than 0.101 MPa to produce an intermediate product stream comprising ACN and an ammonia recovery stream comprising ammonia, and purifying the intermediate product stream to produce a purified product stream comprising greater than 95 wt % aminocapronitrile.

Embodiment 2: an embodiment of embodiment 1, wherein the separating is conducted at a pressure ranging from 0.101 MPa to 0.120 MPa.

Embodiment 3: an embodiment of embodiment 1 or 2, wherein the separating is conducted at a temperature at the top of the column ranging from 60° C. to 90° C.

Embodiment 4: and embodiment of any of embodiments 1-3, wherein the separating is conducted at a temperature at the top of the column ranging from 75° C. to 80° C.

Embodiment 5: an embodiment of any of embodiments 1-4, wherein the separating comprises quenching the first crude product to produce a vapor ammonia recovery stream; a liquid ammonia recovery stream; and a first intermediate product stream.

Embodiment 6: an embodiment of embodiment 5, wherein the vapor ammonia recovery stream comprises greater than 95 wt % ammonia and less than 5 wt % water; the liquid ammonia recovery stream comprises greater than 20 wt % ammonia; and the intermediate product stream comprises heavies and greater than 40 wt % aminocapronitrile.

Embodiment 7: an embodiment of embodiment 5 or 6, further comprising compressing the vapor ammonia recovery stream, preferably via a centrifugal compressor, to form a compressed vapor ammonia recovery stream.

Embodiment 8: an embodiment of any of embodiments 5-7, further comprising separating the liquid ammonia recovery stream to produce a supplemental ammonia recovery stream comprising ammonia and a waste stream.

Embodiment 9: an embodiment of any one of embodiments 5-8, further comprising recycling at least a portion of the vapor ammonia stream, the liquid ammonia recovery stream, and/or supplemental ammonia recovery stream to the ammonia feed stream or to the contacting step.

Embodiment 10: an embodiment of any one of embodiments 1-9, wherein the purifying comprises separating the intermediate product stream to produce a first purified product stream and a crude column bottoms.

Embodiment 11: an embodiment of embodiment 10, wherein the first purified product stream comprises greater than 95 wt. % aminocapronitrile and less than 5 wt. % of impurities comprising cyclohexanone, cyclohexenone, n-hexanenitrile, 5-hexenenitrile, aminocaproic acid, caprolactam, caprolactam dimers caprolactam oligomers, or n-phenylacetamide, or combinations thereof.

Embodiment 12: an embodiment of embodiment 10 or 11, further comprising separating the first purified product stream to form a second purified product stream comprising greater than 95 wt % aminocapronitrile, preferably greater than 99 wt % aminocapronitrile.

Embodiment 13: an embodiment of any one of embodiments 10-12, further comprising separating the crude column bottoms to produce a distillate caprolactam recycle stream comprising greater than 95 wt % caprolactam and heavy byproducts stream containing organic impurities and less than 10 wt % caprolactam.

Embodiment 14: an embodiment of embodiment 13, further comprising recycling the caprolactam recycle stream to the caprolactam feed stream or to the reaction step.

Embodiment 15: an embodiment of any of embodiments 1-14, wherein the process is a continuous process and wherein the process operates at a production rate greater than 100 million pounds per year.

Embodiment 16: an embodiment of any of embodiments 1-15, wherein the caprolactam feed stream comprises less than 99 wt. % caprolactam and greater than 1 wt. % impurities.

Embodiment 17: an embodiment of any of embodiments 1-16, wherein the contacting is conducted in a reactor, preferably a fixed bed reactor.

Embodiment 18: an embodiment of embodiment 12, wherein the second purified product stream is taken as a side-draw.

Embodiment 19: a process for converting caprolactam to aminocapronitrile (ACN), the process comprising: contacting a caprolactam feed stream with ammonia to produce a first crude product stream; separating the first crude product stream via continuous distillation in one or more columns at a pressure less than 100 kPa to produce an intermediate product stream comprising ACN and a caprolactam recovery stream comprising caprolactam; and purifying the intermediate product stream to produce a purified product stream comprising greater than 98 wt. % ACN.

Embodiment 20: the process of Embodiment 19, wherein the residence time in any one column is less than 180 minutes.

Embodiment 21: the process of Embodiments 19 or Embodiment 20, wherein the recovery of ACN in the intermediate product stream is greater than 70%.

Embodiment 22: The process of Embodiments 19-21, wherein the recovery of caprolactam in the caprolactam recovery stream is greater than 90%.

Embodiment 23: A process for converting caprolactam to aminocapronitrile, the process comprising: contacting a caprolactam feed stream with ammonia to produce a first crude product stream; separating the first crude product stream via a continuous process to produce an intermediate product stream comprising aminocapronitrile and a recovery stream comprising ammonia, caprolactam, or a combination thereof; and purifying the intermediate product stream to produce a purified product stream comprising greater than 95 wt % aminocapronitrile.

Embodiment 24: The process of Embodiment 23, wherein the continuous process comprises continuous distillation in one or more columns.

Embodiment 25: The process of Embodiment 23 or Embodiment 24, wherein the separating is conducted at a pressure ranging from 0.1 kPa to 100 kPa.

Embodiment 26: The process of Embodiments 23-25, wherein the temperature at the bottom of the one or more distillation columns during the separating ranges from 150° C. to 180° C.

Embodiment 27: The process of Embodiments 23-26, wherein the reflux ratio during the separating ranges from 1:1 to 10:1.

Embodiment 28: The process of Embodiments 23-27, wherein the purifying comprises separating the intermediate product stream to produce a first purified product stream and a crude column bottoms.

Embodiment 29: The process of Embodiment 28, wherein the first purified product stream comprises greater than 95 wt. % aminocapronitrile and less than 5 wt. % of impurities comprising cyclohexanone, cyclohexenone, n-hexanenitrile, 5-hexenenitrile, aminocaproic acid, caprolactam, caprolactam dimers caprolactam oligomers, or n-phenylacetamide, or combinations thereof.

Embodiment 30: The process of Embodiment 28 or Embodiment 29, further comprising separating the first purified product stream to form a second purified product stream comprising greater than 95 wt % aminocapronitrile, preferably greater than 99 wt % aminocapronitrile.

Embodiment 31: The process of Embodiments 28-30, further comprising separating the crude column bottoms to produce a distillate caprolactam recycle stream comprising greater than 95 wt % caprolactam and heavy byproducts stream containing organic impurities and less than 10 wt % caprolactam.

Embodiment 32: The process of Embodiments 1-31, wherein the caprolactam feed stream comprises less than 99 wt. % caprolactam and greater than 1 wt. % impurities.

Embodiment 33: The process of Embodiments 1-32, wherein the contacting is conducted in a reactor, preferably a fixed bed reactor.

1. A process for converting caprolactam to aminocapronitrile, the process comprising:
contacting a caprolactam feed stream with ammonia to produce a first crude product stream;
separating the first crude product stream via a continuous process to produce an intermediate product stream comprising aminocapronitrile and a recovery stream comprising ammonia, caprolactam, or a combination thereof; and
purifying the intermediate product stream to produce a purified product stream comprising greater than 95 wt % aminocapronitrile.

2. The process of claim 1, wherein the continuous process comprises continuous distillation.

3. The process of claim 1, wherein the separating is conducted at a pressure ranging from 0.1 kPa to 100 kPa.

4. The process of claim 1, wherein the temperature at the bottom of the one or more distillation columns during the separating ranges from 150° C. to 180° C.

5. The process of claim 1, wherein the reflux ratio during the separating ranges from 1:1 to 10:1.

6. The process of claim 1, wherein the purifying comprises separating the intermediate product stream to produce a first purified product stream and a crude column bottoms.

7. The process of claim 6, wherein the first purified product stream comprises greater than 95 wt. % aminocapronitrile and less than 5 wt. % of impurities comprising cyclohexanone, cyclohexenone, n-hexanenitrile, 5-hexenenitrile, aminocaproic acid, caprolactam, caprolactam dimers caprolactam oligomers, or n-phenylacetamide, or combinations thereof.

8. The process of claim 6, further comprising separating the first purified product stream to form a second purified product stream comprising greater than 95 wt % aminocapronitrile, preferably greater than 99 wt % aminocapronitrile.

9. The process of claim 6, further comprising separating the crude column bottoms to produce a distillate caprolactam recycle stream comprising greater than 95 wt % caprolactam and heavy byproducts stream containing organic impurities and less than 10 wt % caprolactam.

10. The process of claim 1, wherein the caprolactam feed stream comprises less than 99 wt. % caprolactam and greater than 1 wt. % impurities.

11. The process of claim 1, wherein the contacting is conducted in a reactor, preferably a fixed bed reactor.

12. A process for converting caprolactam to aminocapronitrile, the process comprising:
contacting a caprolactam feed stream with ammonia to produce a first crude product stream;
separating the first crude product stream via continuous distillation in one or more columns at a pressure less than 100 kPa to produce an intermediate product stream comprising ACN and a caprolactam recovery stream comprising caprolactam; and
purifying the intermediate product stream to produce a purified product stream comprising greater than 98 wt % aminocapronitrile.

13. The process of claim 12, wherein the residence time in any one column is less than 450 minutes.

14. The process of claim 13, wherein the residence time in any one column is less than 180 minutes.

15. The process of claim 12, wherein the reflux ratio during the separating ranges from 1:1 to 10:1.

16. The process of claim 15, wherein the reflux ratio during the separating ranges from 1:1 to 3:1.

17. The process of claim 12, wherein the temperature at the bottom of the distillation column bottom temperature during the separating ranges from 150° C. to 180° C.

18. The process of claim 12, wherein the recovery of aminocapronitrile in the intermediate product stream is greater than 70%.

19. The process of claim 12, wherein the recovery of caprolactam in the caprolactam recovery stream is greater than 90%.

20. The process of claim 12, wherein the contacting comprises a feed rate ranging from 1 klb/hr to 250 klb/hr.
